(19) United States
(12) Patent Application Publication
(10) Pub. No.: US 2024/0201022 A1
(43) Pub. Date: Jun. 20, 2024

(54) TEMPERATURE DETECTOR FOR A HEAT SENSITIVE MATERIAL

(71) Applicant: Caretech Services Pty Ltd, West Perth (AU)

(72) Inventor: David WEARNE, West Perth (AU)

(21) Appl. No.: 18/286,406

(22) PCT Filed: Nov. 16, 2022
(86) PCT No.: PCT/AU2022/051370

§ 371 Date: Oct. 11, 2023

(30) Foreign Application Priority Data
Nov. 17, 2021 (AU) 2021903708

(51) Int. Cl. G01K 3/00; A61M 5/44; G01K 1/024; G01N 25/18

(52) U.S. Cl. CPC G01K 3/005; A61M 5/44; G01K 1/024; G01N 25/18

(57) ABSTRACT

A temperature detector for a heat sensitive product includes a carbon or silicon containing material having a heat transfer property variable with temperature, the carbon or silicon containing material being located proximate to a heat sensitive material; a sensor for measuring the heat transfer property of the carbon or silicon containing material; and a signal generator that provides a signal reflective of the heat transfer property of the carbon or silicon containing material as measured by the sensor and a temperature indication for the heat sensitive product.

### TECHNICAL FIELD

This invention relates to a temperature detector and, in particular, to a temperature detector which may be used for detecting whether a heat sensitive material or product, for example a vaccine, other medicament or food or beverage, remains within determined temperature limits for safety, efficacy or aesthetic reasons.

### BACKGROUND ART

The following discussion of the background art is intended to facilitate an understanding of the present invention only. The discussion is not an acknowledgement or admission that any of the material referred to is or was part of the common general knowledge as at the priority date of the application.

Viral diseases are a major cause of ill health and death throughout the world. The 2020-2022 COVID 19 pandemic is an example of a viral disease affecting large populations and causing a high number of deaths throughout the world. Vaccines to address this issue are under active development with candidates still to be selected. Leading vaccine candidates appear, without limitation, to be under development by AstraZeneca/University of Oxford, Pfizer, Moderna and the Serum Institute of India.

It is no exaggeration to observe that, throughout the world, many billions of doses of vaccines will be required. Against a backdrop of potential low efficacy—perhaps 40-50% in some countries—many doses will have to be administered at many locations. A reliable supply chain is critical to achieving the vaccination objective.

It is to be understood that supply chain issues also apply to other pharmaceuticals, e.g. oncological pharmaceuticals, and that problems similar to those for vaccines also exist, albeit on a smaller scale though no less critically to those impacted.

In that respect, the present Applicant has developed a system (the SIP System™) accepting data from a portable therapeutic platform for a therapeutic procedure comprising the steps of a) providing at least one platform comprising at least one therapeutically effective item required to conduct the therapeutic procedure and an electronic communications device for receiving and transmitting information, such as temperature, epidemiological or zootic information, about an environment surrounding and including the platform to a computer system including a processor. The processor processes this information and initiates an automated control response to said processed information. The automated control response may take a number of forms in each case using the transmitted information to develop higher quality therapeutic platforms and better quality public health responses.

The Applicant's SIP system has, in one implementation, a number of advantages in supply chain assurance and may be used in relation to transporting vaccines or other pharmaceuticals subject to cold chain requirements. Further detail about the SIP system is contained in the Applicant's U.S. Pat. No. 11,456,067, the contents of which are hereby incorporated herein by reference. If convenient to enable information transmission, an app may be used to provide an interface between the electronic communications device and computer system. Therapeutic platforms in the form of packs may be used for diagnosis of the viral disease, or other conditions. Such packs may include therapeutically effective items in a container suitable for use with the system whether packets, boxes or otherwise. Packs may include electronic devices such as wearable devices which include a therapeutically effective item for diagnosis or treatment of a patient.

Vaccines, medicaments or other therapeutically effective items to respond to the viral or other disease may be sourced from a virtual stockpile (for example co-ordinated by an NGO or government authority) which is managed on the basis of information received from therapeutic packs deployed with viral diagnostic tests, the information being processed by the computer system to provide quantitative information about the scale of a viral disease outbreak in both epidemiological terms and also in a form that can be processed, including through the use of artificial intelligence and machine learning, for example for the control of manufacturing processes for items required to respond to the viral disease outbreak. Such information may similarly be processed to identify potential therapies for a disease, for example through application of bioinformatics.

The SIP system may advantageously incorporate and/or manage software as a medical device (SAMD), for example as defined by the International Medical Device Regulators Forum (IMDRF), as a therapeutically effective item. In this context, SAMD may include devices as specified by the Australian TGA—a member of IMDRF—in the online document ‘Regulation of software based medical devices’, 17 Aug. 2022 and available at www.tga.gov.au, the contents of which document are hereby incorporated by reference. Other regulators, which are members of IMRDF, would apply similar definitions to SAMD as the Australian TGA.

For example, the SIP system could be used for diagnosis of a respiratory viral disease through processing of a patient's voice signals with a control response being initiated proportionate to the voice test result. In another form, the pack could automatically report a patient's test results, for example the result of a mandatory RAT or other test, again with an automated control response—for example controlling scale and location of pack manufacture—being initiated proportionate to an aggregation of RAT test results from a population, which may be geographically segmented to optimise the automated control response. In the latter case, the automated control response would typically be aimed at a population, rather than at an individual.

The SIP system is not limited in its application to viral diseases; cancer, for example, is another class of conditions amenable to management by the Applicant's SIP system). Diabetes is yet another example.

Returning to vaccines, a problem that requires resolution is maintaining some forms of vaccine within a determined low temperature range—typically in the range 2 to 8° C. though lower temperatures may be required dependent on vaccine form (mRNA or other)—throughout the supply chain. This ‘cold chain’ is necessary to maintain vaccine viability, efficacy and safety at the point of administration to a patient. It is therefore necessary to determine whether a vaccine leaves its determined low temperature range during transport. If this can be determined, by a suitable sensor which could be called a cold chain detector, then corrective action can be taken to address the issues leading to breaking cold chain and the detriments that can arise from that. Other medicaments also require cold chain management, these medicaments including—for example—oncological or anti-cancer drugs, insulin and asthma medications.

Current cold chain detectors or sensors, which provide an electronic signal indicative of temperature (as opposed to passive sensors), are expensive which can currently make cold chain monitoring impractical—particularly at scale—with detriment arising from that as described above. It is not even clear, from the Applicant's researches, that a cold chain detector suitable for all territories, many of which may be cost sensitive and subject to limited government funding for vaccine delivery is currently available.

Further, while cold chain detection for vaccines and medicaments is important, it would also be useful to determine when other types of heat sensitive products—whether medical or in the food and beverage or other industries—have temperature falling outside a determined temperature range for safe and effective storage and/or transport.

It is against the above background that the present invention has been developed.

### SUMMARY OF INVENTION

It is an object of the present invention to provide a temperature detector and system that provides information about when a product is outside a determined temperature range.

With this object in view, the present invention provides a temperature detector for measuring temperature of a heat sensitive product comprising:
(a) a carbon or silicon containing material having a heat transfer property variable with temperature, said carbon or silicon containing material being located proximate to a heat sensitive material;
(b) a sensor for measuring the heat transfer property of said carbon or silicon containing material; and
(c) a signal generator that provides a signal reflective of said heat transfer property of said carbon or silicon containing material as measured by the sensor and a temperature indication for said heat sensitive product.

Preferably, the heat transfer property is thermal conductivity or thermal conductance. “Thermal conductivity” is defined as the rate at which heat passes through the carbon or silicon containing material, expressed as the amount of heat that flows per unit time through a unit area of the carbon or silicon containing material with a temperature gradient of one degree per unit distance (with the units being W/m-K or W/m-° C.). “Thermal conductance” is defined as the rate at which heat passes through the carbon or silicon containing material, expressed as the amount of heat that passes through the carbon or silicon containing material given one unit area of the carbon or silicon containing material and a temperature gradient through the thickness of the material (with the units being W/m/K or W/m/° C.).

Advantageously, the material is a carbon containing material containing a, desirably stable, member of the fullerene family of molecules with the C20, C60 and C70 fullerenes being examples. Particle size may be mm size or greater. Endohedral fullerenes may be preferred as multi-functional materials that may be used as temperature detectors as well as for highly accurate tracking as described below. Fullerenes have a cage like structure and may be known as Buckminster fullerene or simply ‘bucky-balls’. Endohedral fullerenes have a species—typically an atom such as N, P, a rare earth or lanthanide, or molecule such as H₂O—located within the fullerene cage. C20 fullerenes have a thermal conductivity approximately linearly proportional to temperature in the ambient temperature range. Single crystal C60 fullerenes have a thermal conductivity that changes significantly at about −13° C. Single crystal C70 fullerenes have a thermal conductivity that changes significantly at about 27° C. A mixture of fullerenes, for example C60 and C70 fullerenes, may be contained within the carbon containing material. The endohedral fullerene may be supported on a solid substrate such as a silica wafer.

As to silicon containing material, a suitable material may be silica used for wafers and integrated circuits and chips. Silica wafers have an almost constant rate of variation in thermal conductivity over the temperature range of interest in ‘cold chain’ management, and as shown in FIG. 3, allowing a determination of temperature of the heat sensitive material.

The carbon or silicon containing material is located proximate the heat sensitive material which may include a vaccine or other medicament (including a heat sensitive intermediate to the production of a medicament) requiring to be stored and/or transported within a determined temperature range. Proximate location would desirably involve location of the carbon or silicon containing material being within a pack containing the heat sensitive material rather than in a bulk container holding a plurality of packs.

A change in a property, such as thermal conductivity or thermal conductance, of the carbon or silicon containing material, advantageously a fullerene as above described, may be used as an indication of the temperature of the heat sensitive material and—in particular—when the temperature falls outside the determined temperature range for maintaining stability of the heat sensitive material. A precise measurement of temperature is not always required, it may be sufficient for the temperature detector to indicate that the heat sensitive material has a temperature—as reflected by measured heat transfer property (e.g. thermal conductivity)—that is outside the bounds of the determined temperature range. The temperature detector can therefore be used as a cold chain monitor, sending a signal reflective of whether temperature of the heat sensitive material is within the determined temperature range with the required cold chain being maintained. If the determined temperature range is not maintained, the reasons for this can be investigated and corrective action taken to restore cold chain integrity. It will be appreciated that where critically important materials, such as temperature sensitive vaccines, are concerned, it is important to minimise wastage (which has run at 20% or more in India's eVIN vaccination programme) or unsafe administration so corrective actions may be of the utmost importance.

Preferably, the temperature detector may be associated with a timer which determines duration for which the heat sensitive material remains outside the determined temperature range.

The temperature detector may be used alternatively or additionally to a passive temperature monitor for the heat sensitive material—such as the colour changing materials typically applied to vaccines which provides an indication of whether a vaccine is still safe and effective for administration to patients. It will be appreciated that passive temperature monitors do not provide the functionality described herein.

A carbon containing material may also be used as a tracking indicator for the heat sensitive material. Fullerenes, such as those endohedral fullerenes functioning as “atomic clocks” as described in U.S. Pat. No. 8,217,724, the contents of which are hereby incorporated herein by reference, Such miniature atomic clocks may be included, along with other components, within methods and apparatus for accurately calculating time that can receive, process and communicate information to enable locating, identifying and tracking physical assets, for example vaccines and other heat sensitive materials as described in U.S. Pat. No. 10,310,053, assigned to LocatorX, Inc, the contents of which are hereby incorporated herein by reference. In another aspect of the invention, the methods and apparatus of U.S. Pat. No. 10,310,053 may be used to locate, identify and track therapeutic packs or items included within the packs as described below though, in one option, may exclude the temperature detector of the present invention.

The heat sensitive material may be a therapeutic item included within a portable therapeutic platform comprising:
a) at least one therapeutic item required to conduct the therapeutic procedure; and
b) an electronic communications device for receiving and transmitting information about an environment surrounding and including the platform. One form of platform is a pack.

In another aspect, the present invention provides a system for managing the use of a portable therapeutic platform for a therapeutic procedure comprising the steps of:
a) providing at least one platform comprising at least one heat sensitive therapeutic item required to conduct the therapeutic procedure and an electronic communications device for receiving and transmitting information about an environment surrounding and including the platform;
b) transferring information, including temperature information, about the environment surrounding and including at least one platform between the electronic communications device and a computer system including a processor which processes said information; and
c) initiating a control response to said processed information
wherein temperature of said heat sensitive therapeutic item, such as a vaccine or medicament such as an oncological drug, is detected by a temperature detector for a heat sensitive product comprising:
   a carbon or silicon containing material having a heat transfer property variable with temperature, said carbon or silicon containing material being located proximate to said heat sensitive therapeutic item;
   a sensor for measuring the heat transfer property of said carbon or silicon containing material; and
   a signal generator that provides a signal reflective of said measured heat transfer property of said carbon or silicon containing material as measured by the sensor and a temperature indication for said heat sensitive therapeutic item.

The platform may comprise a therapeutic pack as described in the Applicant's U.S. Pat. No. 11,456,067, incorporated by reference. Information about the environment, including temperature, patient or zoonotic data, may be transferred directly or through an interface such as an app. This information may be processed by the processor and used to build a virtual or remote stockpile of therapeutic items with a focus on acquiring or developing effective therapeutic items to effectively respond, for example, to a viral disease epidemic or pandemic.

Preferably, the system allows tracking of the therapeutic platform (or pack). Tracking may be implemented by RFID, NFC, GPS, Bluetooth or wireless communications protocol. Most preferably, the system may enable tracking using a tracking indicator in the form of a fullerene, or endohedral fullerene, in the form of an atomic clock, for example following the Global Resource Locator (GRL) methodology (using triangulation and trilateration) as described in U.S. Pat. No. 10,310,053, the contents of which are incorporated herein by reference for all purposes. Where the fullerene can also be used as a temperature indicator, as described above, this provides a cost advantage as the cost of a temperature indicator can be included within the cost of a tracking indicator.

The temperature detector and systems of the present invention provide an indication of whether heat sensitive materials have temperature outside determined temperature limits for safe and effective storage and transport. The temperature detector can be used, without limitation, to monitor cold chain performance of vaccines (including freeze dried vaccines), other medicaments (such as pharmaceuticals including anti-cancer or oncological drugs) and reagents for diagnostic or use in other therapeutic procedures. In any event, corrective action may be taken to address any factors leading to breach of cold chain. In this regard, a cold chain monitoring system using the temperature detector may assist insurers and re-insurers to minimise losses in potentially expensive therapeutic items due to cold chain breaches.

Advantageously, the temperature detector can also be used as a tracking indicator suitable for monitoring supply chain performance and indicating the location and provenance of the heat sensitive material as well as the location at which any excursion of the temperature of the heat sensitive material from determined temperature range—e.g. cold chain temperature range—occurs further assisting corrective actions to be taken to avoid wastage of the heat sensitive material. While vaccines and other heat sensitive medicaments (including heat sensitive chemical intermediates to the production of medicaments) are of interest, other heat sensitive materials include food and beverage materials (for example including olive oil, wine and other foodstuffs that require maintenance within a cold chain in terms of transport temperature and operation of cooling/heating equipment to maintain transport temperature) and tracking the temperature of such materials through the supply chain may also be advantageous for safety, efficacy, aesthetic or other reasons.

In one embodiment relating to food or beverage transport, there is provided a system, as above described, where the food is, for example, olive oil and temperature information is used to control heating or cooling of olive oil to a predetermined temperature during transport. The same methodology may be applied to other food and beverage products.

### BRIEF DESCRIPTION OF THE DRAWINGS AND EXAMPLES

Further features of the temperature detector and systems of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a block diagram schematically showing a temperature detector for a therapeutic pack according to one embodiment of the invention.

FIG. 2 is a plot of thermal conductivity versus temperature for a C20 fullerene.

FIG. 3 is a plot of thermal conductivity versus temperature for a silicon wafer.

### DESCRIPTION OF PREFERRED EMBODIMENTS

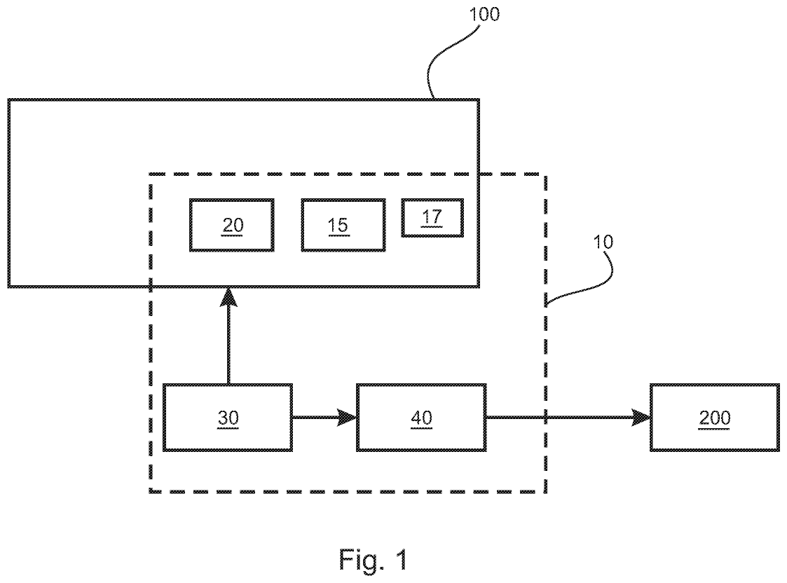

Referring to FIG. 1, there is shown a temperature detector 10 for a heat sensitive product 15, such as a vaccine or pharmaceutical (including an oncological drug) contained within a therapeutic pack 100. Temperature detector 10 comprises a carbon or silicon containing material 20 having a detectable heat transfer property variable with temperature. The carbon or silicon containing material 20 is located proximate to the heat sensitive product 15. Taking a vaccine as an example of a heat sensitive material contained within a prefilled syringe 15 manufactured from polymer in embodiments by a Rommelag BFS (i.e. blow-fill-seal) machine with further information being available at www.rommelag.com, the contents of which are hereby incorporated herein by reference, the vaccine may be responsive to a viral disease such as the COVID-19 disease caused by the SARS-Cov-2 virus. The vaccine will lose efficacy and safety above a prescribed temperature as exemplified below, whether below −13° C. or outside the range 2 to 8° C. dependent on vaccine biochemistry. It will be understood that exemplification of prefilled syringes is not intended to be limiting. Ampoules, vials or other vessels or medical devices (including wearable electronic devices) containing medicaments requiring temperature control or cold chain management may have temperature detected in the described manner.

The temperature detector 10 further includes a thermal conductivity sensor 30 for measuring thermal conductivity of the carbon or silicon containing material 20; and a signal generator 40 that provides a signal reflective of the thermal conductivity of the carbon or silicon containing material as measured by sensor 30 and a temperature indication for said heat sensitive material. Transmitter 17 enables this signal to be transmitted (together with a unique ID code for therapeutic pack 100) to a computing device forming part of a control system 200 which can process this information and initiate a control response as described below and in the Applicant's U.S. Pat. No. 11,456,067, incorporated by reference. An app may be used as an interface if convenient for information transfer between therapeutic pack 100 and control system 200. Transmitter 17 may be supported by further electronic communication means as described in U.S. Pat. No. 11,456,067 as required.

It will be understood that, where thermal conductivity of the carbon or silicon containing material 20 is a function of temperature, i.e. not being independent of temperature, a measurement of thermal conductivity can—through relation to a plot of thermal conductivity versus temperature—be related to a temperature value. While such measurement may be precise, this is not always necessary. It may be sufficient, for purposes of cold chain detection, for example, to indicate an approximate temperature or even an excursion beyond a temperature limit, whether that be 2 to 8° C. or lower temperature, for example −13° C. (where a C60 single crystal fullerene may be used as carbon containing material 20). In another example, a heat sensitive product, for example a food or beverage product, such as olive oil, may unacceptably degrade—possibly through microbial action or oxidation—at temperature over 27° C. (where a C70 single crystal fullerene may be used as carbon containing material 20).

Thermal conductivity sensors 30 may be of a range of types as known to the person skilled in the art of thermal conductivity sensing. For example, in embodiments, a portable thermal conductivity meter such as available from Thermtest Instruments Europe under the trade name TLS 100 may be used. Laser flash apparatus as available from Netzsch under the trade mark LFA-457 Microflash® may be used. BioMEMS-based thermal probes, for example as described in Liang X M et al., Micro-sensors for Determination of Thermal Conductivity of Biomaterials and Solutions, in Kulacki, F (ed), Handbook of Thermal Conductivity of Biomaterials and Solutions, Springer, (2017), 1-28 (Cham. https://doi.org/10.1007/978-3-319-32003-8_74-1), the contents of which are hereby incorporated herein by reference, may also be applicable. It will be understood that thermal conductivity or thermal conductance has been measured for a range of fullerene molecules in the quantum computing context and illustrative and non-limiting examples for use according to the present invention are provided below.

The time for which the heat sensitive material remains at a temperature outside the permitted temperature limit, for example 2 to 8° C. or lower temperature, for example −13° C. or over 27° ° C. may also be determined by relating a thermal conductivity outside the required range as measured by thermal conductivity sensor 30 to time as measured by a timer or clock.

In the following examples, therapeutic pack 100 includes a portion of carbon containing material 20, in the form of a fullerene molecules having thermal conductivity variable as a function of temperature.

Carbon materials 20, for purposes of current exemplification are as follows:
Example 1 Single crystal C60 fullerene
Example 2 C20 fullerene, Hexa-C20
Example 3 Single crystal C70 fullerene

### Example 1

A therapeutic pack 100, held in a temperature controlled space (such as a cold store forming part of a logistical supply chain) including a refrigeration system, may include a carbon material 20 in the form of mm size substantially pure C60 fullerene crystals, for example prepared by sublimation to remove solvent (following prior extraction from an arc method produced fullerene enriched soot, for example by high-performance liquid chromatography) and as described in Tea, N H et al., Thermal conductivity of C60 and C70 fullerene crystals, Applied Physics A, 56, 219-225 (1993) (“Tea et al.”), the contents of which are hereby incorporated herein by reference. Carbon material 20 is located proximate a prefilled syringe 15 containing the heat sensitive vaccine which is to be maintained at a temperature below −13° C.

For such a C60 fullerene, thermal conductivity (k)—as measured by a static method as described in Tea et al. incorporated herein by reference—is about 0.4 W/m K at room temperature with k slightly linearly declining to about −13° C. where there is a 25% jump in k and an approximately linear increase in k at lower temperatures.

With thermal conductivity determined by the Tea et al. method, the detection of a 25% fall in thermal conductivity of the C60 fullerene held within the temperature controlled space acts as a temperature detection signal indicating that the prefilled syringe 15 and the vaccine contained therein has a temperature greater than −13° C., above the determined temperature limit for the heat sensitive material.

Thermal conductivity may be measured every 30 to 60 seconds (near continuously)—dependent on saturation of the thermal voltage—and logged in a memory of a computing system 200 to which the thermal conductivity sensor 30 for therapeutic pack 100 and 15 is electronically and wirelessly connected.

The computer system 200 includes a timer which indicates the time for which the prefilled syringe 15 has temperature above −13° C. or, on the contrary, on sensing of a jump in thermal conductivity of 25% or more, indicates that the heat sensitive vaccine is within the determined temperature limits, i.e. below −13° C. The computer system 200, via transmitter 17, may also determine and/or provide location information for the therapeutic pack 100, whether through the GRL methodology, for example as described in U.S. Pat. No. 10,310,053 incorporated herein by reference, a GPS tracking methodology or otherwise. A manufacturer or supplier of prefilled syringe 15 may thereby be provided with information about temperature and location of the prefilled syringe 15 via computer system 200.

Methodology as employed in the Applicant's U.S. Pat. No. 11,456,067 may be used to effect an automated control response to the vaccine temperature rising above the −13° C. temperature limit. This may involve the vaccine manufacturer supplying further vaccine, preferably after the reason for the temperature controlled space exceeding the −13° C. temperature limit being determined and addressed, for example by rectifying a fault in the refrigeration system for the temperature controlled space though, in other embodiments, temperature sensing would be at the vaccine pack level. The vaccine may be sourced from a virtual stockpile managed on the basis of information received from therapeutic packs deployed with viral diagnostic tests and with diagnostic results processed by computer system 200. The information may be processed in a manner described in the Applicant's U.S. Pat. No. 11,456,067, for example using artificial intelligence and machine learning algorithms as generally described in their application in the healthcare sector (for example in Devi, K G et al (Eds), Machine Learning and Deep Learning Techniques for Medical Science, CRC Press (2022); and Lidstromer, N and Ashrafian, (Eds.), Artificial Intelligence in Medicine, Springer Nature Switzerland AG, (2022) the contents of which are hereby incorporated herein by reference), to manage a virtual stockpile including the prefilled syringes 15 and other items required to respond to the viral disease.

### Example 2

A therapeutic pack 100, held in a temperature controlled space (such as a cold store forming part of a logistical supply chain) including a refrigeration system, could include a carbon material 20 in the form of Hexa-C20 fullerene-based sheet with thermal conductivity properties as predicted by Shen, Y et al., A 2D C20 fullerene-based sheet with ultrahigh thermal conductivity, Nanoscale, 10, 6099-6104, (2018) (“Shen et al.”), the contents of which are hereby incorporated herein by reference. Carbon material 20 is located proximate an ampoule 15 containing the heat sensitive vaccine which is to be maintained at a temperature between 2 and 8° C.

With thermal conductivity determined by the Shen et al. model, the detection of thermal conductivity of the C20 sheet held within the temperature controlled space acts as a temperature detection signal indicating the temperature of the ampoule 15 because of the linear relationship between k and temperature over a large temperature range and including the range 2 to 8° C.

Thermal conductivity may be measured, for example, by the method of Tea et al. incorporated herein by reference, every 30 to 60 seconds (near continuously)—dependent on saturation of the thermal voltage—and logged in a memory of a computer system 200 to which the thermal conductivity sensor for therapeutic pack 100 and ampoule 15 is electronically and wirelessly connected.

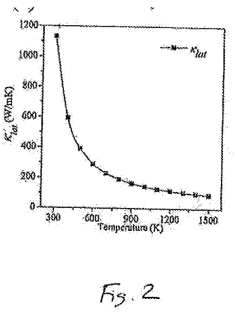

The computer system 200 includes a look up map that relates thermal conductivity (kₗₐₜ) to temperature by the Shen et al. model (as shown in FIG. 2). If detected thermal conductivity rises above that recorded in the look up map for 2° C., a low limit excursion for the carbon material 20 would be indicated. If detected thermal conductivity falls below that recorded in the look up map for 8° C., a high limit excursion for the carbon material 20 would be indicated

The computer system 200 includes a timer which allows capture of the time for which the ampoule 15 and its heat sensitive vaccine has temperature outside the determined temperature limits, i.e. 2 to 8° C.

The computer system 200 may also determine and/or provide location information for the therapeutic pack 100, whether through the GRL methodology, for example as described in U.S. Pat. No. 10,310,053, a GPS tracking methodology or otherwise. A manufacturer or supplier of ampoule 15 may thereby be provided with information about temperature and location of the ampoule 15 via computer system 200. Methodology as employed in the Applicant's U.S. Pat. No. 11,456,067 may be used to effect an automated control response to the vaccine temperature falling outside the determined 2 to 8° C. temperature limit. This may involve the vaccine manufacturer supplying further vaccine, preferably after the reason for the temperature controlled space falling outside the 2 to 8° C. temperature limit being determined and addressed, for example by rectifying a fault in the temperature control system for the temperature controlled space though in other embodiments temperature sensing would be at the vaccine pack level. The ampoules 15 may be sourced from a virtual stockpile managed on the basis of information received from therapeutic packs deployed with viral diagnostic tests and processed by computer system 200. The information may be processed in a manner described in the Applicant's U.S. Pat. No. 11,456,067, for example using artificial intelligence and machine learning algorithms as generally described in their application in the healthcare sector (for example in Devi, K G et al (Eds), Machine Learning and Deep Learning Techniques for Medical Science, CRC Press (2022); and Lidstromer, N and Ashrafian, (Eds.), Artificial Intelligence in Medicine, Springer Nature Switzerland AG, (2022) the contents of which are hereby incorporated herein by reference), to manage a virtual stockpile including the vaccine, ampoules, syringes and other items required to respond to the viral disease. While ampoule 15 here contains a vaccine, it will be understood that the ampoule could also be filled with other heat sensitive medical preparations or heat sensitive intermediates to the production of medicaments.

### Example 3

A therapeutic pack 100, held in a temperature controlled space (such as a cold store forming part of a logistical supply chain) including a refrigeration system, may include a carbon material 20 in the form of mm size substantially pure C70 fullerene crystals, for example prepared by sublimation to remove solvent (following prior extraction from an arc method produced fullerene enriched soot, for example by high-performance liquid chromatography) and as described in Tea, N H et al., Thermal conductivity of C60 and C70 fullerene crystals, Applied Physics A, 56, 219-225 (1993) (“Tea et al.”), the contents of which are hereby incorporated herein by reference. Carbon material 20 is located proximate an ampoule 15 containing the heat sensitive vaccine which is to be maintained at a temperature below −13° C.

For such a C70 fullerene, thermal conductance—as measured by a static method as described in Tea et al. incorporated herein by reference—falls sharply but linearly in the range −10 to about 27° C. above which there is a 25% jump. Thermal conductance rather than thermal conductivity is relied on here because thermal conductivity information for C70 fullerene, especially solvent containing C70 fullerene, may not be reproducible.

With thermal conductance determined by the Tea et al. method, the thermal conductance is compared with a look up map programmed into the computing device that relates thermal conductance to temperature based on the Tea et al data (see FIG. 10 of Tea et al.). If detected thermal conductance falls below that recorded in the look up map for 2° C., a low limit excursion for the carbon material 20 would be indicated. If detected thermal conductance falls below that recorded in the look up map for 8° C., a high limit excursion for the carbon material 20 would be indicated.

A computing device of computer system 200 includes a timer which allows capture of the time for which the ampoule 15 and its heat sensitive vaccine or oncological drug has temperature outside the determined temperature limits, i.e. 2 to 8° C.

The computer system 200 may also determine and/or provide location information for the therapeutic pack 100, whether through the GRL methodology, for example as described in U.S. Pat. No. 10,310,053 incorporated herein by reference, a GPS tracking methodology or otherwise. A manufacturer or supplier of ampoule 15 may thereby be provided with information about temperature and location of the ampoule 15. Methodology as employed in the Applicant's U.S. Pat. No. 11,456,067 may be used to effect a control response to the vaccine temperature falling outside the determined 2 to 8° C. temperature limit. This may involve the vaccine manufacturer supplying further vaccine, preferably after the reason for the temperature controlled space falling outside the 2 to 8° C. temperature limit being determined and addressed, for example by rectifying a fault in the temperature control system for the temperature controlled space. The ampoules 15 may be sourced from a virtual stockpile managed on the basis of information received from therapeutic packs deployed with viral diagnostic tests and processed by computer system 200. The information may be processed in a manner described in the Applicant's U.S. Pat. No. 11,456,067, for example using artificial intelligence and machine learning algorithms as generally described in their application in the healthcare sector (for example in Devi, K G et al (Eds), Machine Learning and Deep Learning Techniques for Medical Science, CRC Press (2022); and Lidstromer, N and Ashrafian, (Eds.), Artificial Intelligence in Medicine, Springer Nature Switzerland AG, (2022) the contents of which are hereby incorporated herein by reference), to manage a virtual stockpile including the vaccine, ampoules 15, syringes and other items required to respond to the viral disease, e.g. COVID-19 caused by the SARS-Cov-2 virus.

In other embodiments, the therapeutic pack 100 may include a portion of silicon containing material 20, in the form of a silicon wafer having thermal conductivity variable as a function of temperature.

### Example 4

A therapeutic pack 100, held in a temperature controlled space (such as a cold store forming part of a logistical supply chain) including a refrigeration system, may include a silicon material 20 in the form of a silicon wafer, the contents of which are hereby incorporated herein by reference. Silicon wafer 20, which has a density of 2.4 g/cm³ and a thickness of 0.7 mm, is located proximate an ampoule 15 containing the heat sensitive vaccine which is to be maintained at a temperature in the range 2 to 8° C.

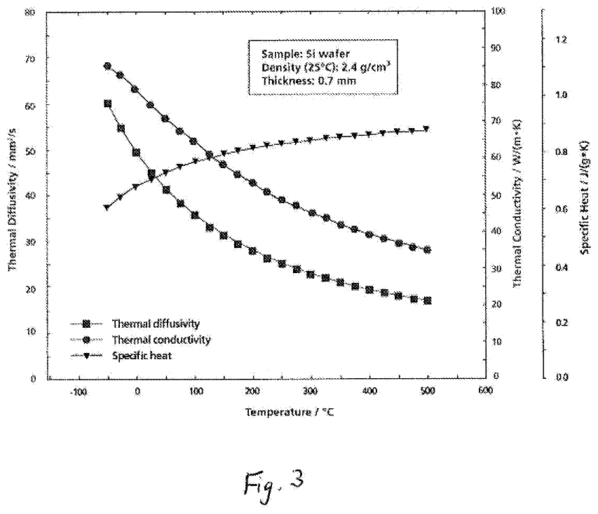

For a silicon wafer, thermal conductivity—as measured by a laser flash apparatus as available from Netzsch under the trade mark LFA-457 Microflash®—falls linearly at a more or less constant rate in the range −10 to about 27° C. as shown in FIG. 3.

With thermal conductivity, as determined by the laser flash method, the thermal conductivity is compared with a look up map programmed into the computing device that relates thermal conductivity to temperature based on the data shown in FIG. 3. If detected thermal conductivity falls below that recorded in the look up map for 2° C., a low limit excursion for the heat sensitive material 20 would be indicated. If detected thermal conductivity falls below that recorded in the look up map for 8° C., a high limit excursion for the heat sensitive material 20 would be indicated.

A computing device of computer system 200 includes a timer which allows capture of the time for which the ampoule 15 and its heat sensitive vaccine or oncological drug has temperature outside the determined temperature limits, i.e. 2 to 8° C.

The computer system 200 may also determine and/or provide location information for the therapeutic pack 100, whether through the GRL methodology, for example as described in U.S. Pat. No. 10,310,053, a GPS tracking methodology or otherwise. A manufacturer or supplier of ampoule 15 may thereby be provided with information about temperature and location of the ampoule 15. Methodology as employed in the Applicant's U.S. Pat. No. 11,456,067 may be used to effect a control response to the vaccine temperature falling outside the determined 2 to 8° C. temperature limit. This may involve the vaccine manufacturer supplying further vaccine, preferably after the reason for the temperature controlled space falling outside the 2 to 8° C. temperature limit being determined and addressed, for example by rectifying a fault in the temperature control system for the temperature controlled space. The ampoules 15 may be sourced from a virtual stockpile managed on the basis of information received from therapeutic packs deployed with viral diagnostic tests and processed by computer system 200. The information may be processed in a manner described in the Applicant's U.S. Pat. No. 11,456,067, for example using artificial intelligence and machine learning algorithms as generally described in their application in the healthcare sector (for example in Devi, K G et al (Eds), Machine Learning and Deep Learning Techniques for Medical Science, CRC Press (2022); and Lidstromer, N and Ashrafian, (Eds.), Artificial Intelligence in Medicine, Springer Nature Switzerland AG, (2022) the contents of which are hereby incorporated herein by reference), to manage a virtual stockpile including the vaccine, ampoules 15, syringes and other items required to respond to the viral disease, e.g. COVID-19 caused by the SARS-Cov-2 virus.

Modifications and variations to the temperature detector and systems of the present invention may be apparent to the skilled reader of this disclosure. Such modifications and variations are deemed within the scope of the present invention. For example, it will be understood that the heat sensitive material need not necessarily be a vaccine. It could be another medicament, such as an oncological drug, insulin or other. The heat sensitive material could also be a food or beverage such as olive oil or wine.

Throughout this specification, unless the context requires otherwise, the word “comprise” or variations such as “comprises” or “comprising”, will be understood to imply the inclusion of a stated integer or group of integers but not the exclusion of any other integer or group of integers.

1. A cold chain monitor comprising:
a carbon or silicon containing material having a heat transfer property variable with temperature, the carbon or silicon containing material being located proximate to a heat sensitive material to be maintained in a determined temperature range;
a sensor for measuring the heat transfer property of the carbon or silicon containing material; and
a signal generator that provides a signal reflective of the heat transfer property of the carbon or silicon containing material as measured by the sensor and an electronic indication for indicating when the temperature of the heat sensitive product falls outside the determined temperature range.

2. The cold chain monitor of claim 1, wherein the heat transfer property is thermal conductivity or thermal conductance.

3. The cold chain monitor of claim 2, wherein the material is a carbon containing material being a member of a fullerene family of molecules.

4. (canceled)

5. The cold chain monitor of claim 3, wherein the member of the fullerene family of molecules is selected from an endohedral fullerene selected from the group consisting of C20, C60 and C70 fullerenes.

6. The cold chain monitor of claim 5, wherein a mixture of fullerenes is contained within the carbon containing material.

7. The cold chain monitor of claim 6, wherein the mixture of fullerenes includes C60 and C70 fullerenes.

8. The cold chain monitor of claim 5, wherein the endohedral fullerene is supported on a solid substrate.

9. The cold chain monitor of claim 1, wherein the silicon containing material is a silica wafer.

10. The cold chain monitor of claim 2, wherein the heat sensitive material includes a vaccine or other medicament requiring to be stored and/or transported within the determined temperature range.

11. The cold chain monitor of claim 10, configured to send a signal reflective of whether temperature of the heat sensitive material is within the determined temperature range.

12. (canceled)

13. The cold chain monitor of claim 7, wherein the carbon containing material is also used as a tracking indicator for the heat sensitive material.

14. The cold chain monitor of claim 7, wherein the heat sensitive material is a therapeutic item included within a portable therapeutic platform comprising:
at least one therapeutic item required to conduct a therapeutic procedure; and
an electronic communications device for receiving and transmitting information about an environment surrounding and including the platform.

15. A system for managing use of a portable therapeutic platform for a therapeutic procedure comprising the steps of:
providing at least one platform comprising at least one heat sensitive therapeutic item required to conduct the therapeutic procedure and an electronic communications device for receiving and transmitting information about an environment surrounding and including the platform;
transferring information, including temperature information, about the environment surrounding and including at least one platform between the electronic communications device and a computer system including a processor which processes the information; and
initiating a control response to the processed information,
wherein a heat sensitive material is to be maintained in a determined temperature range and a temperature of the heat sensitive therapeutic item is detected by a temperature detector for a heat sensitive product comprising:
a carbon or silicon containing material having a heat transfer property variable with temperature, the carbon or silicon containing material being located proximate to the heat sensitive therapeutic item;
a sensor for measuring the heat transfer property of the carbon or silicon containing material; and
a signal generator that provides a signal reflective of the measured heat transfer property of the carbon or silicon containing material as measured by the sensor and an electronic temperature indication for indicating when the heat sensitive therapeutic item falls outside the determined temperature range.

16. The system of claim 15, wherein the platform comprises a therapeutic pack.

17. The system of claim 15, wherein the information about the environment, including temperature data is transferred directly or through an interface including an app.

18. The system of claim 17, wherein the system enables tracking using a tracking indicator in a form of a member of an endohedral fullerene family of molecules.

19. The system of claim 18, wherein the endohedral fullerene is in a form of an atomic clock.

20. (canceled)

21. The system of claim 19, wherein the member of the endohedral fullerene family is selected from the group consisting of C20, C60 and C70 fullerenes.

22. The system of claim 21, wherein the carbon containing material contains a mixture of fullerenes.

23. The system of claim 22, wherein the mixture of fullerenes includes C60 and C70 fullerenes.

24. The system of claim 15, wherein the information is processed by the processor and used to build a virtual or remote stockpile of therapeutic items.

25. (canceled)

26. The system of claim 15, comprising monitoring of cold chain performance of vaccines and medicaments.

27. The system of claim 15, comprising monitoring of temperature of a food or beverage during transport.

28. The system of claim 27, wherein the food is olive oil and temperature information is used to control heating or cooling of olive oil to a predetermined temperature during transport.
